(19) United States
(12) Patent Application Publication
(10) Pub. No.: US 2019/0167204 A1
(43) Pub. Date: Jun. 6, 2019

(54) WORSENING HEART FAILURE DETECTION BASED ON PATIENT DEMOGRAPHIC CLUSTERS

(71) Applicant: Cardiac Pacemakers, Inc., St. Paul, MN (US)

(72) Inventors: Jianjun Yuan, Minneapolis, MN (US); Qi An, Blaine, MN (US); Rezwan Ahmed, Arden Hills, MN (US); Pramodsingh Hirasingh Thakur, Woodbury, MN (US)

(21) Appl. No.: 16/180,937

(22) Filed: Nov. 5, 2018

Related U.S. Application Data
(60) Provisional application No. 62/595,531, filed on Dec. 6, 2017.

(51) Int. Cl. A61B 5/00; A61B 5/0205; G16H 50/20; G16H 10/60; G16H 20/10

(52) U.S. Cl. CPC A61B 5/7275; A61B 5/0205; A61B 5/4836; A61B 5/02405; G16H 10/60; G16H 20/10; G16H 50/20

(57) ABSTRACT

Systems and methods for monitoring patients for risk of worsening heart failure (WHF) are discussed. A patient management system includes a receiver circuit to receive a heart failure phenotype of the patient including patient demographic information, The system may include a classifier circuit to classify the patient into one of a plurality of phenotypes based on the received heart failure phenotype. The plurality of phenotypes are each represented by multi-dimensional categorized demographics. A detector circuit may detect a WHF event from a physiologic signal using the classified phenotype. The system may include a therapy circuit to deliver or adjust a heart failure therapy in response to the detected WHF event.

### CLAIM OF PRIORITY

This application claims the benefit of priority under 35 U.S.C. § 119(e) of U.S. Provisional Patent Application Ser. No. 62/595,531, filed on Dec. 6, 2017, which is herein incorporated by reference in its entirety.

### TECHNICAL FIELD

This document relates generally to medical devices, and more particularly, to systems, devices and methods for assessing patient risk of worsening heart failure.

### BACKGROUND

Congestive heart failure (CHF) is a leading cause of death in the United States and globally. CHF is the loss of pumping power of the heart, and may affect left heart, right heart, or both sides of the heart, and result in the inability to deliver enough blood to meet the demands of peripheral tissues. CHF patients typically have enlarged heart with weakened cardiac muscles, resulting in reduced contractility and poor cardiac output of blood. CHF may be treated by drug therapy, or by an implantable medical device (IMD) such as for providing electrostirnulation therapy, Although usually a chronic condition, CHF may occur suddenly.

Some IMDs are capable of monitoring CHF patients and detect events leading to worsening heart failure (WHF). These IMDs may include sensors to sense physiologic signals from a patient. Frequent patient monitoring may help reduce heart failure hospitalization. Identification of patient at an elevated risk of developing WHF, such as heart failure decompensation, may help ensure timely treatment and improve prognosis and patient outcome, identifying and safely managing the patients at elevated risk of WHF may avoid unnecessary medical interventions, hospitalization, and thereby reduce healthcare cost.

An may contain electronic circuitry, such as a pulse generator, to generate and deliver electrostimulation to excitable tissues or organs, such as a heart. The electrostimulation may help restore or improve a CHF patient's cardiac performance, or rectify cardiac arrhythmias. One example of such electrostimulation therapy is resynchronization therapy (CRT) for correcting cardiac dyssynchrony in CHF patients.

### SUMMARY

Frequent monitoring of CHF patients and timely detection of intrathoracic fluid accumulation or other events indicative of heart failure decompensation status may help prevent WHF in CHF patients, hence reducing cost associated with heart failure hospitalization.

Ambulatory medical devices for monitoring heart failure patient may include implantable medical devices (IMD), subcutaneous medical devices, wearable medical devices or other external medical devices. An ambulatory medical device may be coupled to one or more physiologic sensors to sense electrical activity and mechanical function of the heart. The ambulatory medical device may optionally deliver therapy, such as electrical stimulation pulses, to the patient to restore or improve patient cardiac function. Some of these devices may provide diagnostic features, such as using transthoracic impedance or other sensor signals. For example, fluid accumulation in the lungs decreases the transthoracic impedance due to the lower resistivity of the fluid than air in the lungs. The fluid accumulation may also elevate ventricular filling pressure, resulting in a louder S3 heart sound. Additionally, fluid accumulation in the lungs may irritate the pulmonary system and leads to decrease in tidal volume and increase in respiratory rate.

Identification of patient at an elevated risk of WHF may help ensure timely intervention such as device therapy or drug therapy, thereby improving the prognosis and patient outcome. On the other hand, identifying and safely managing patients with low risk of WHF may avoid unnecessary medical interventions, thereby reducing healthcare cost. Desired performance of WHF risk stratification may include one or more of a high sensitivity, a high specificity, a high positive predictive value (PPV), or a negative predictive value (NPV). The sensitivity represents an accuracy of identifying patients with relatively a high risk of WHF The specificity represents an accuracy of identifying patients with relatively a low risk of WHF. Conventionally, WHF risk stratification has been focused on patient demographic data such as age, sex, race, or pre-disposing risk factors such as hypertension, coronary artery disease, or prior heart failure hospitalization. However, factors such as difference of medical conditions across patients and/or disease progression within a patient may also contribute to patient risk of developing a future WHF event. The present inventors have recognized that there remains a considerable need of systems and methods that may accurately identify CHF patients with an elevated risk of WHF, such as developing a heart failure decompensation event.

This document discusses, among other things, a patient management system for assessing patient risk of WHF. In an embodiment, a medical system may receive from the patient a heart failure phenotype, which may include patient demographic information, medical history information, or medication information. The system includes a classifier circuit to classify the patient into one of a plurality of phenotypes based on the received patient heart failure phenotype. The plurality of phenotypes are each represented by multi-dimensional categorized demographics. A detector circuit may detect a WHF event from a physiologic signal using the classified phenotype. The system may include a therapy circuit to deliver or adjust a heart failure therapy in response to the detected WHF event.

Example 1 is a system for detecting worsening heart failure (WHF) in a patient. The system comprises a signal receiver configured to receive a physiologic signal from the patient, a phenotype receiver configured to receive a heart failure phenotype of the patient including patient demographic information, and a processor circuit. The processor circuit includes a classifier circuit configured to classify the patient into one of a plurality of phenotypes based on the received heart failure phenotype, and a detector circuit configured to detect a WHF event using the sensed physiologic signal and the classified phenotype. The plurality of phenotypes each may be represented by multi-dimensional categorized demographics.

In Example 2, the subject matter of Example 1 optionally includes the plurality of phenotypes each of which may further include medical history information,

In Example 3, the subject matter of any one or more of Examples 1-2 optionally includes the plurality of phenotypes each of which may further include medication information.

In Example 4, the subject matter of any one or more of Examples 1-3 optionally includes the received heart failure phenotype that may further include medical history or medication information of the patient. The classifier circuit may be configured to classify the patient into one of the plurality of phenotypes in response to a change in the medical history or medication of the patient.

In Example 5, the subject matter of any one or more of Examples 1-4 optionally includes a storage device that may be configured to store a correspondence between the plurality of phenotypes and the corresponding multi-dimensional categorized demographics. The classifier circuit may be configured to classify the patient into one of the plurality of phenotypes using the stored correspondence.

In Example 6, the subject matter of any one or more of Examples 1-5 optionally includes the classifier circuit that may be configured to determine similarity metrics between the received heart failure phenotype and each of the plurality of phenotypes, and to classify the patient into one of the plurality of phenotypes based on the similarity metrics.

In Example 7, the subject matter of any one or more of Examples 1-6 optionally includes the classifier circuit that may be configured to compute a patient phenotype score using the received heart failure phenotype, and to classify the patient into one of the plurality of phenotypes based on the computed patient phenotype score.

In Example 8, the subject matter of Example 7 optionally includes the classifier circuit that may be configured to compute the patient phenotype score using a combination of numerical values respectively assigned to the received patient demographic information.

In Example 9, the subject matter of any one or more of Examples 1-8 optionally includes the detector circuit that may be configured to identify a detection algorithm based on the classified phenotype, and to detect the WHF event using the identified detection algorithm and the sensed physiologic signal.

In Example 10, the subject matter of any one or more of Examples 1-9 optionally includes the detector circuit that may be configured to compute a composite signal metric using the sensed physiologic signal, and to detect the WHF event using the composite signal metric.

In Example 11, the subject matter of Example 10 optionally includes the detector circuit that may be configured to adjust a threshold value based on the classified phenotype threshold value, and to detect the WHF event using a comparison of the composite signal metric to the adjusted threshold value.

In Example 12, the subject matter of any one or more of Examples 10-11 optionally includes the detector circuit that may be configured to: generate a plurality of signal metrics from the sensed physiologic signal; assign weight factors to the plurality of signal metrics based on the classified phenotype; and compute the composite signal metric using a weighted combination of the plurality of the signal metrics respectively scaled by the assigned weight factors. The weight factor assignment may include one or more of increasing a weight factor to a respiration rate metric if the classified phenotype includes an attribute of significant shortness of breath, increasing a weight factor to a heart rate metric if the classified phenotype includes an attribute of palpitation, or increasing a weight factor to a total thoracic impedance metric if the classified phenotype includes an attribute of edema.

In Example 13, the subject matter of any one or more of Examples 1-12 optionally includes a sensor circuit that may be configured to selectively sense physiologic signal based on the classified phenotype. The detector circuit may be configured to detect a WHF event using the selectively sensed physiologic signal.

In Example 14, the subject matter of any one or more of Examples 1-13 optionally includes an output circuit that may be configured to generate an alert of the detected WHF event.

In Example 15, the subject matter of any one or more of Examples 1-14 optionally includes a therapy circuit that may be configured to generate and deliver a heart failure therapy in response to the detection of the WHF event.

Example 16 is a method for detecting worsening heart failure (WHF) a patient using a medical system. The method comprises steps of: receiving a physiologic signal from the patient; receiving a heart failure phenotype of the patient including patient demographic information; and classifying the patient into one of a plurality of phenotypes based on the received heart failure phenotype, the plurality of phenotypes each represented by multi-dimensional categorized demographics; and detecting a WHF event using the sensed physiologic signal and the classified phenotype.

In Example 17, the subject matter of Example 16 optionally includes delivering a heart failure therapy in response to the detection of the WHF event.

In Example 18, the subject matter of Example 16 optionally includes the received heart failure phenotype including medical history or medication information of the patient. The classifier circuit may be configured to classify the patient into one of the plurality of phenotypes in response to a change in the medical history or medication of the patient.

In Example 19, the subject matter of Example 16 optionally includes determining similarity metrics between the received heart failure phenotype and each of the plurality of phenotypes. The classification of the patient into one of the plurality of phenotypes may be based on the similarity metrics.

In Example 20, the subject matter of Example 16 optionally includes computing a patient phenotype score using the received heart failure phenotype. The classification of the patient into one of the plurality of phenotypes may be based on the computed patient phenotype score.

In Example 21, the subject matter of Example 16 optionally includes computing a composite signal metric using the sensed physiologic signal. The detection of the WHF event may be based on the composite signal metric.

In Example 22, the subject matter of Example 21 optionally includes adjusting a threshold value based on the classified phenotype threshold value. The detection of the WHF event may include using a comparison of the composite signal metric to the adjusted threshold value.

In Example 23, the subject matter of Example 21 optionally includes generating a plurality of signal metrics from the sensed physiologic signal, and assigning weight factors to the plurality of signal metrics based on the classified phenotype. The computation of the composite signal metric may include a weighted combination of the plurality of the signal metrics respectively scaled by the assigned weight factors. The weight factor assignment may include one or more of increasing a weight factor to a respiration rate metric if the classified phenotype includes an attribute of significant shortness of breath, increasing a weight factor to a heart rate metric if the classified phenotype includes an attribute of palpitation, or increasing a weight factor to a total thoracic impedance metric if the classified phenotype includes an attribute of edema.

Various embodiments described herein may help improve the medical technology of device-based heart failure patient management, particularly computerized detection of progression of a chronic disease such as WHF. It has been recognized that patients with different heart failure phenotypes (e.g., demographics, medical history, or medication) may exhibit different physiologic reactions to the progression of heart failure. The phenotype-based WHF detection as discussed in this document involves automatic adjustment of detection algorithms or detection parameters based on the patient heart failure phenotype. The patient phenotype may be classified into one of pre-determined clusters each represented by a known phenotype. When the patient medical condition changes, the patient may be reclassified into a different pre-determined phenotype; and the WHF detection algorithm may be automatically adjusted to adapt to the new phenotype. Conventionally, WHF detection algorithms may be pre-determined or static, and are not sufficiently individualized to accommodate patient changing medical conditions. A change of WHF detection algorithm may require human intervention such as manually programming a device. The present phenotype-based WHF detection may substantially automate the process of dynamically adjusting WHF detection algorithms based on patient changing medical conditions, and help reduce false positive rate and improve accuracy of WHF detection, Additionally, the classification of patient heart failure phenotype and a change from one classified phenotype to another are useful heart failure diagnostics that indicate progression of patient heart failure status. Therefore, systems, devices, and methods discussed in this document may improve the technology of computerized WHF assessment.

With the improved WHF risk assessment, the systems and methods discussed herein may identify patients at WHF risk timely and reliably yet at little to no additional cost. Such improvement in heart failure patient management may reduce hospitalization and healthcare costs associated with patient management. The systems, devices, and methods discussed in this document may also allow for more efficient device memory usage, such as by storing WHF risk indicators that are clinically more relevant to WHF risk stratification. As fewer false positive detections of WHF events are provided, device battery life may be extended; fewer unnecessary drugs and procedures may be scheduled, prescribed, or provided. Therapy titration, such as electrostimulation parameter adjustment, based on the generated WHF risk, may not only improve therapy efficacy and patient outcome, but may also save device power. As such, overall system cost savings may be realized,

Although the discussion in this document focuses WHF risk assessment, this is meant only by way of example and not limitation. It is within the contemplation of the inventors, and within the scope of this document, that the systems, devices, and methods discussed herein may also be used to detect, and alert occurrence of, cardiac arrhythmias, syncope, respiratory disease, or renal dysfunctions, among other medical conditions. Additionally, although systems and methods are described as being operated or exercised by clinicians, the entire discussion herein applies equally to organizations, including hospitals, clinics, and laboratories, and other individuals or interests, such as researchers, scientists, universities, and governmental agencies, seeking access to the patient data.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient monitor system and portions of an environment in which the system may operate.

FIG. 2 illustrates generally an example of a heart failure monitor system configured to detect a WHF event from a patient.

FIGS. 3A-3B illustrate generally examples of a mapping from various phenotypes to corresponding detection configurations.

FIG. 4 illustrates generally a diagram of computing a phenotype score for the patient heart failure phenotype.

FIG. 5 illustrates generally an example of a method for detecting WHF in a patient based on phenotype classification.

FIG. 6 illustrates generally a block diagram of an example machine upon which any one or more of the techniques discussed herein may perform.

### DETAILED DESCRIPTION

Disclosed herein are systems, devices, and methods for monitoring a patient for WHF. A medical system may receive a heart failure phenotype from the patient, which includes patient demographic information. The system includes a classifier circuit to classify the patient into one of a plurality of phenotypes based on the received heart failure phenotype. The plurality of phenotypes are each represented by multi-dimensional categorized demographics. A detector circuit may detect a WHF event from a physiologic signal using the classified phenotype. The system may include a therapy circuit to deliver or adjust a heart failure therapy in response to the detected WHF event.

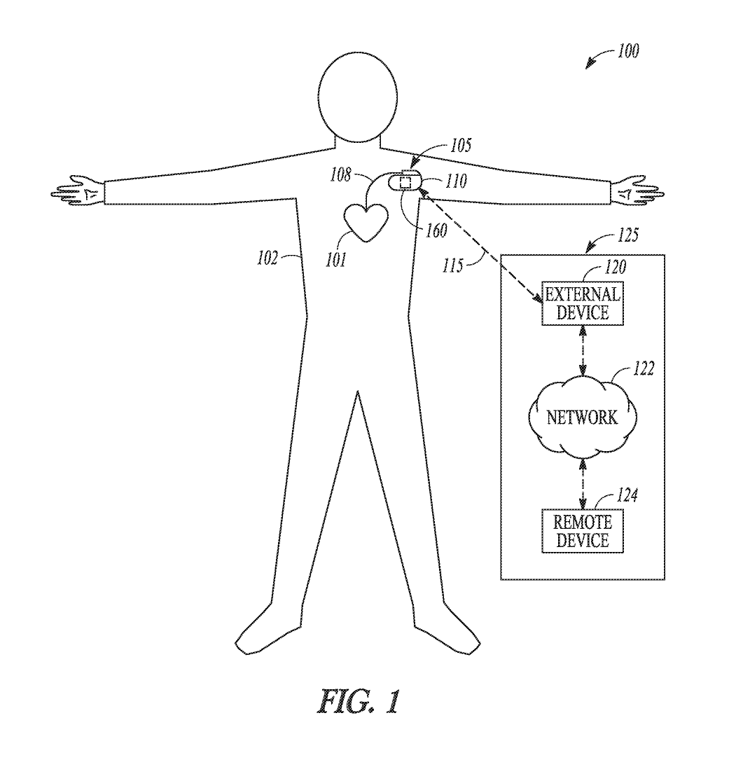

FIG. 1 illustrates generally an example of a patient monitor system 100 and portions of an environment in which the system 100 may operate. The patient monitor system 100 may chronically monitor a patient 102 to assess patient risk of developing WHF. Portions of the system 100 may be ambulatory. Portions of the system 100 may be disposed in a patient home or office, a hospital, a clinic, or a physician's office.

As illustrated in FIG. 1, the patient monitor system 100 may include an ambulatory system 105 associated with the patient 102, an external system 125, and a telemetry link 115 providing for communication between the ambulatory system 105 and the external system 125. The ambulatory system 105 may include an ambulatory medical device (AMD) 110. In an example, the AMD 110 may be an implantable: device subcutaneously implanted in a chest, abdomen, or other parts of the patient 102. Examples of the implantable device may include, but are not limited to, pacemakers, pacemaker/defibrillators, cardiac resynchronization therapy (CRT) devices, cardiac remodeling control therapy (RCT) devices, neuromodulators, drug delivery devices, biological therapy devices, diagnostic devices such as cardiac monitors or loop recorders, or patient monitors, among others. The AMD 110 may include a subcutaneous medical device such as a subcutaneous monitor or diagnostic device, external monitoring or therapeutic medical devices such as automatic external defibrillators (AEDs) or Holter monitors, or wearable medical devices such as patch-based devices, smart wearables, or smart accessories.

By way of example and not limitation, the AMD 110 may be coupled to a lead system 108. The lead system 108 may include one or more transvenously, subcutaneously, or non-invasively placed leads or catheters. Each lead or catheter may include one or more electrodes. The arrangements and uses of the lead system 108 and the associated electrodes may be determined using the patient need and the capability of the AMD 110. The associated electrodes on the lead system 108 may be positioned at the patient's thorax or abdomen to sense a physiologic signal indicative of cardiac activity, or physiologic responses to diagnostic or therapeutic stimulations to a target tissue. By way of example and not limitation, and as illustrated in FIG. 1, the lead system 108 may be surgically inserted into, or positioned on the surface of, a heart 101. The electrodes on the lead system 108 may be positioned on a portion of a heart 101, such as a right atrium (RA), a right ventricle (RV), a left atrium (LA), or a left ventricle (LV), or any tissue between or near the heart portions. In some examples, the lead system 108 and the associated electrodes may alternatively be positioned on other parts of the body to sense a physiologic signal containing information about patient heart rate or pulse rate. In an example, the ambulatory system 105 may include one or more leadless sensors not being tethered to the AMD 110 via the lead system 108. The leadless ambulatory sensors may be configured to sense a physiologic signal and wirelessly communicate with the AMD 110.

The AMD 110 may include a hermetically sealed can that houses one or more of a sensing circuit, a control circuit, a communication circuit, and a battery, among other components. The sensing circuit may sense a physiologic signal, such as by using a physiologic sensor or the electrodes associated with the lead system 108. The physiologic signals may contain information about patient physiologic response to a precipitating event associated with onset of a future WHF event. The physiologic signal may represent changes in patient hemodynamic status. Examples of the physiologic signal may include one or more of electrocardiogram, intracardiac electrogram, arrhythmia, heart rate, heart rate variability, intrathoracic impedance, intracardiac impedance, arterial pressure, pulmonary artery pressure, left atrial pressure, right ventricular (RV) pressure, left ventricular (LV) coronary pressure, coronary blood temperature, blood oxygen saturation, one or more heart sounds, intracardiac acceleration, physical activity or exertion level, physiologic response to activity, posture, respiratory rate, tidal volume, respiratory sounds, body weight, or body temperature.

The AMD 110 may include a heart failure detector circuit 160 configured to detect a WHF event, The heart failure detector circuit 160 may include a sensor circuit to receive a physiologic signal from the patient. The heart failure detector circuit 160 may be communicatively coupled to an input device to receive information about patient heart failure phenotype. The heart failure phenotype is a collection of patient attributes related to heart failure, which may include patient vital signs, multi-dimensional patient demographic information, medical history, dietary and physical activity patterns, weight, and heart failure comorbid conditions, clinical and lab assessments, among others. In heart failure patients, the heart failure phenotypes may vary from patient to patient. In addition to the inter-patient phenotype variation, a patient's heart failure phenotype may vary when patient medical condition changes, such as developing new comorbidity, taking new medication, or receiving new treatment. The heart failure detector circuit 160 takes into account the inter-patient difference in phenotypes and the intra-patient variation in phenotype over time, and classifies the patient into one of a plurality of pre-determined heart failure phenotypes based on the received heart failure phenotype. The pre-determined heart failure phenotypes may each be associated with a corresponding detection algorithm. The heart failure detector circuit 160 may detect the a WHF event using the sensed physiologic signal and a phenotype-indicated WHF detection algorithm.

The AMD 110 may include a therapy unit that may generate and deliver a therapy to the patient. The therapy may be preventive (e.g., to prevent development into a hill-blown), or therapeutic (e.g., to treat heart failure or alleviate complications) in nature, and may modify, restore, or improve patient physiologic functionalities. Examples of the therapy may include electrical, magnetic, or other forms of therapy. In some examples, the AMD 110 may include a drug delivery system such as a drug infusion pump device to deliver drug therapy to the patient. In some examples, the AMD 110 may monitor patient physiologic responses to the delivered to assess the efficacy of the therapy.

The external system 125 may include a dedicated hardware/software system such as a programmer, a remote server-based patient management system, or alternatively a system defined predominantly by software running on a standard personal computer. The external system 125 may manage the patient 102 through the AMD 110 connected to the external system 125 via a communication link 115. This may include, for example, programming the AMD 110 to perform one or more of acquiring physiologic data, performing at least one self-diagnostic test (such as for a device operational status), analyzing the physiologic data to generate a WHF risk. indicator, or optionally delivering or adjusting a therapy to the patient 102. The external system 125 may communicate with the AMD 110 via the communication link 115. The device data received by the external system 125 may include real-time or stored physiologic data from the patient 102, diagnostic data, responses to therapies delivered to the patient 102, or device operational status of the AMI) 110 (e.g., battery status and lead impedance). The communication link 115 may be an inductive telemetry link, a capacitive telemetry link, or a radio-frequency (RF) telemetry link, or wireless telemetry based on, for example, “strong” Bluetooth or IEEE 802.11 wireless fidelity “WiFi” interfacing standards. Other configurations and combinations of patient data source interfacing are possible.

By way of example and not limitation, the external system 125 may include an external device 120 in proximity of the AMD 110, and a remote device 124 in a location relatively distant from the AMD 110 in communication with the external device 120 via a telecommunication network 122. Examples of the external device 120 may include a programmer device. The network 122 may provide wired or wireless interconnectivity. In an example, the network 122 may be based on the Transmission Control Protocol/Internet Protocol (TCP/IP) network communication specification, although other types or combinations of networking implementations are possible. Similarly, other network topologies and arrangements are possible.

The remote device 124 may include a centralized server acting as a central hub for collected patient data storage and analysis. The patient data may include data collected by the AMD 110, and other data acquisition sensors or devices associated with the patient 102. The server may be configured as a uni-, multi- or distributed computing and processing system. In an example, the remote device 124 may include a data processor configured to perform heart failure detection or risk stratification using respiration data received by the AMD 110. Computationally intensive algorithms, such as machine-learning algorithms, may be implemented in the remote device 124 to process the data retrospectively to detect WHF or analyze patient WHF risk. The remote device 124 may generate an alert notification. The alert notifications may include a Web page update, phone or pager call, E-mail, SMS, text Or “Instant” message, as well as a message to the patient and a simultaneous direct notification to emergency services and to the clinician. Other alert notifications are possible.

One or more of the external device 120 or the remote device 124 may output the WHF detection or the WHF risk to a system user such as the patient or a clinician. The external device 120 or the remote device 124 may include respective display for displaying the physiologic data acquired by the AMD 110. The physiologic data may be presented in a table, a chart, a diagram, or any other types of textual, tabular, or graphical presentation formats. The external device 120 or the remote device 124 may include a printer for printing hard copies of signals and information related to the generation of WHF risk indicator. The presentation of the output information may include audio or other media format. In an example, the output unit 254 may generate alerts, alarms, emergency calls, or other forms of warnings to signal the system user about the WHF detection or risk. The clinician may review, perform further analysis, or adjudicate the WHF detection or WHF risk. The WHF detection or the WHF risk, optionally along with the data acquired by the AMD 110 and other data acquisition sensors or devices, may be output to a process such as an instance of a computer program executable in a microprocessor. In an example, the process may include an automated generation of recommendations for initiating or adjusting a therapy, or a recommendation for further diagnostic test or treatment.

Portions of the AMD 110 or the external system 125 may be implemented using hardware, software, firmware, or combinations thereof. Portions of the AMD 110 or the external system 125 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

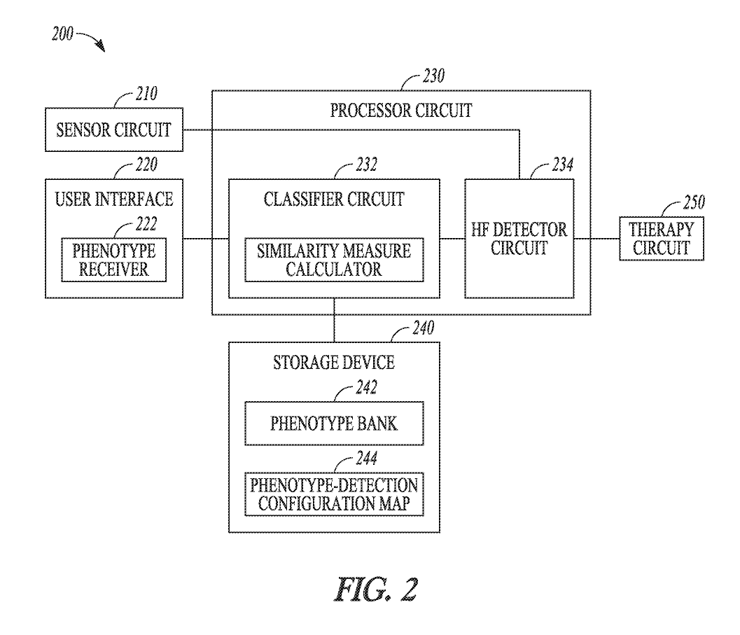

FIG. 2 illustrates generally an example of a heart failure monitor system 200 that may be configured to detect a WHF event from a patient. At least a portion of the heart failure monitor system 200 may be implemented in the AMD 110, the external system 125 such as one or more of the external device 120 or the remote device 124, or distributed between the AMD 110 and the external system 125. The heart failure monitor system 200 may include one or more of a sensor circuit 210, a user interface 220, a processor circuit 230, a storage device 240, and an optional therapy circuit 250 for delivering a heart failure therapy.

The sensor circuit 210 may include a sense amplifier circuit to sense at least one physiologic signal from a patient. The sensor circuit 210 may be coupled to an implantable, wearable, or otherwise ambulatory sensor or electrodes associated with the patient. The sensor may be incorporated into, or otherwise associated with an ambulatory device such as the AMD 110. Examples of the physiologic signals for detecting the precipitating event may include surface electrocardiography (ECG) sensed from electrodes placed on the body surface, subcutaneous ECG sensed from electrodes placed under the skin, intracardiac electrogram (EGM) sensed from the one or more electrodes on the lead system 108, heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. In some examples, the physiologic signals sensed from a patient may be stored in a storage device, such as an electronic medical record system, and the sensor circuit 210 may be configured to receive a physiologic signal from the storage device in response to a user input or triggered by a specific event. The sensor circuit 210 may include one or more sub-circuits to digitize, filter, or perform other signal conditioning operations on the sensed physiologic signal.

The user interface 220, which may be implemented in the external system 125, includes a phenotype receiver 222 that may receive a heart failure phenotype of the patient. The heart failure phenotype may include patient vital signs, patient demographic information, medical history including prior medical, surgical, or treatment, dietary and physical activity patterns, weight, and heart failure comorbid conditions, clinical assessment, lab assessments such as blood urea nitrogen (BUN) level, thiamine pyrophosphate (TPP) level, or other blood chemistry. Because the patient phenotype may change over time, the user interface 220 may prompt a user to provide an updated phenotype. Alternatively, the phenotype may be automatically updated in response to a triggering event, such as a change in the medical history or medication of the patient.

The user interface 220 may include a display to display a questionnaire, and prompt a user to provide information about patient heart failure phenotype. A user, such as the patient or a clinician, may use a keyboard, an on-screen keyboard, a mouse, a trackball, a touchpad, a touch-screen, or other pointing or navigating devices to enter information about patient heart failure phenotype. In some examples, a user may be prompted to make selections from a plurality of pre-determined heart failure phenotypes. The user interface 220 may receive other user input for programming one or more system components, such as the sensor circuit 210, the classifier circuit 232, the heart failure detector circuit 234, or the therapy circuit 250.

The processor circuit 230 may be configured to detect a WHF event using the sensed physiologic signal based on the received patient heart failure phenotype. The processor circuit 230 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The processor circuit 230 may include circuit sets comprising one or more other circuits or sub-circuits including a classifier circuit 232 and a heart failure detector circuit 234. These circuits or sub-circuits may, either individually or in combination, perform the functions, methods or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The classifier circuit 232 may classify the patient into one of a plurality of heart failure phenotypes based on the received patient heart failure phenotype. In the illustrated example, the classifier circuit 232 may be coupled to a storage device 240 that stores a plurality of pre-determined heart failure phenotypes {P₁, P₂, . . . , P_{N}} in a heart failure phenotype bank 242. The pre-determined heart failure phenotypes may each include one or more patient attributes, such as information about patient demographics, medical history, medication intake and dosage, lab tests, among others. The number and/or types of patient attributes included in the heart failure phenotype may differ from one phenotype to another. The patient attributes included in a phenotype may have a numerical value or a range of numerical values (e.g., age=45-55 years old), or a categorical value (e.g., race=Caucasian). By way of example and not limitation, one or more of the following pre-determined heart failure phenotypes may be included in the heart failure phenotype bank:
P₁={Age=old, Race=Caucasian, Medication=no beta blocker};
P₂={Age=old, Race=Caucasian, Medical History=ischemic, Medication=no beta blocker};
P₃={Age=young, Race=Caucasian, Sex=female, Body Mass Index (BMI)=high, Medical History=non-ischemic, Treatment=no coronary artery bypass grafting (CABG)};
P₄={Age=young, Race=African American, Lab=high TPP level};
P₅={Age=old, Race=Caucasian, Lab=high BUN level, Treatment=heart valve surgery},
P₆={Age=young, Race=Caucasian, Blood Pressure=low, Medical History=No hypertension};

Patients classified into different phenotypes (e.g., one of P₁-P₆) may have different heart failure event rate, represented by the amount of heart failure events within a specified time period (e.g., a month, or several months). For example, patients in phenotype P₁ may experience more frequent heart failure events than patients in phenotype P₃. A heart failure detector, when applied to patients with different phenotypes, may result in different detection performance (e.g., different sensitivity, specificity, positive predictive value, or negative predicative value). For example, while patients in phenotype P₅ may experience a lower heart failure event rate than patients in phenotype P₁, a heart failure detector, when applied to patients in P₅ and P₁, may produce significantly more alerts of heart failure event detections in the patients of phenotype P₅ than patients in phenotype P₁. That is, more false positive detections (thus a lower specificity) may have occurred to patients of phenotype P₅ than patients in phenotype P₁. Adjusting a heart failure detector based on patient phenotype, or choose different heart failure detectors indicated by patient phenotype, may reduce false positive detections or false alerts while maintaining or improving detection sensitivity, thereby improving overall performance of heart event detections in a wide range of patients.

The classifier circuit 232 may search the phenotype bank 242 for a target heart failure phenotype (P*) that matches the received patient heart failure phenotype (Px) using a pattern recognition method. Recognition of the target phenotype may be based on similarity metrics to the pre-determined heart failure phenotypes. In an example, the similarity metric is a distance in the multi-dimensional attribute space. The classifier circuit 232 may identify a target phenotype as one with a shortest distance to the patient heart failure phenotype, that is, d(P*, Px)=min(Pᵢ, Px) for i=1, 2, . . . , N. Examples of the distance metric may include Euclidean distance, Mahalanobis distance, correlation coefficient, or a L1, L2, or infinite norm, among others.

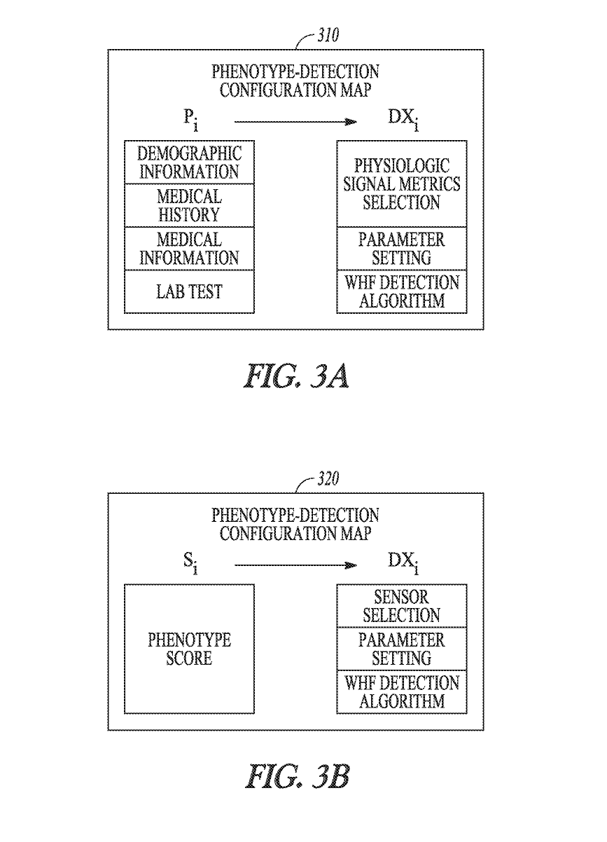

The storage device 240 may further include a phenotype-detection configuration map 244 that associates each of the pre-determined heart failure phenotype (Pᵢ) in the phenotype bank 242 with a detection configuration (DXᵢ). The DXᵢ may include an optimal parameter setting for detecting a WHF event in patients having the same phenotype Pᵢ. In an example, the phenotype-detection configuration map 244 may be constructed based on heart failure phenotypes and the WHF event detection performance information collected from a large patient population. The optimal parameter setting for the phenotype Pᵢ may he determined as one that leads to a WHF event detection performance (e.g., WHF event detection sensitivity, specificity, or positive predictive value) satisfying a specific condition using data collected from patients having the same phenotype Pᵢ. The DXᵢ may additionally or alternatively include a selection of physiologic signal metrics and/or a selection of a WHF detection algorithm for WHF event detection. Using the phenotype-detection configuration map 244, the processor circuit 230 may identify a detection configuration (DX*) corresponding to the target heart failure phenotype (P*). Examples of mapping from a pre-determined phenotype to a detection configuration are discussed below, such as with reference to FIG. 3A.

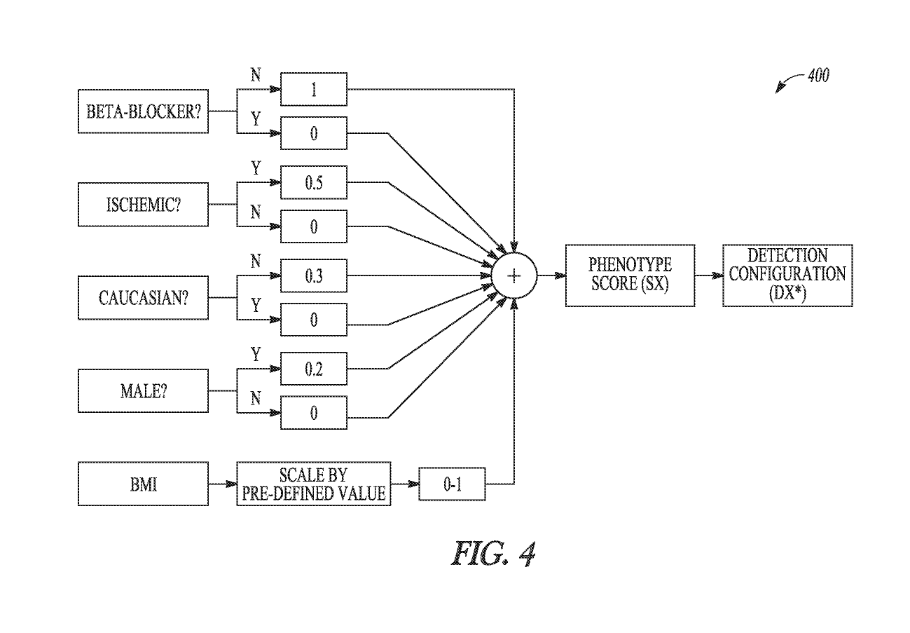

In some examples, the classifier circuit 232 may classify the patient heart failure phenotype without referring to the phenotype bank 242 and recognizing a target phenotype in the phenotype bank 242. The classifier circuit 232 may instead compute a patient phenotype score using attributes of the received patient heart failure phenotype. Each attribute of the patient heart failure phenotype that satisfies a specific condition (e.g., exceeding a threshold, falling within a value range, or being categorized into a specific category) may be assigned an attribute score. The classifier circuit 232 may compute a phenotype score (S_{X}) for the received patient heart failure phenotype (P_{X}), and classify the patient into one of the plurality of phenotypes based on the computed phenotype score. The phenotype-detection configuration map 244 may include a mapping between a phenotype score or a score range (Sᵢ) and a detection configuration (DXᵢ). Using the phenotype-detection configuration map 244, the processor circuit 230 may identify a detection configuration (DX*) corresponding to the phenotype score (S_{X}) computed based on the received patient heart failure phenotype (P_{X}). Examples of the phenotype score-based classification and mapping to detection configuration are discussed below, such as with reference to FIGS. 3B and 4.

The heart failure detector circuit 234 is coupled to the sensor circuit 210 and configured to detect a WHF event using the sensed physiologic signal. In an example, the heart failure detector circuit 234 may further include a signal metric generator that may generate one or more signal metrics from the sensed physiologic signal. The signal metrics may include statistical or morphological features. By way of example and not limitation, the signal metrics may include heart rate, heart rate variability, cardiac activation timings, morphological features from the ECG or EGM, thoracic or cardiac impedance magnitude within a specified frequency range, intensities or timings of S1, S2, S3, or S4 heart sounds, systolic blood pressure, diastolic blood pressure, mean arterial pressure, or timing of a pressure metric with respect to a fiducial point, among others. In various examples, the signal metrics may be trended over time.

The heart failure detector circuit 234 may be coupled to the classifier circuit 232 and the storage device 240, and retrieve the detection configuration (DX*) corresponding to the received patient heart failure phenotype (P*). The heart failure detector circuit 234 may detect a WHF event using the trended signal metrics according to the detection configuration DX*. In an example, the heart failure detector circuit 234 may detect the WHF event by comparing a signal metric to a detection threshold as specified in the detection configuration DX*. In some examples, the WHF detector circuit 234 may generate a composite signal index using a combination of two or more signal metrics derived from the one or more physiologic signals, detect a WHF event and generate a WHF alert when the composite signal index exceeds a detection threshold. The detection threshold and the two or more signal metrics selected for computing the composite signal index may be specified in the detection configuration DX*. In some examples, the WHF detector circuit 234 may process the signal metric trend and generate a predictor trend indicating temporal changes of the signal metric trend. The temporal change may be calculated using a difference between short-term values and baseline values. In an example, the short-term values may include statistical values such as a central tendency of the measurements of the signal metric within a short-term window of a first plurality of days. The baseline values may include statistical values such as a central tendency of the measurements of the signal metric within a long-term window of a second plurality of days preceding the short-term window in time. The parameters used for computing the short-term and long-term value may be specified in the detection configuration DX*. In some examples, the predictor trend may be determined using a linear or nonlinear combination of the relative differences between multiple short-term values corresponding to multiple first time windows and multiple baseline values corresponding to multiple second time windows. The differences may be scaled by respective weight factors which may be based on timing information associated with corresponding multiple short-term window, such as described by Thakur et al., in U.S. Patent Publication 2017/0095160, entitled “PREDICTIONS OF WORSENING HEART FAILURE”, which is herein incorporated by reference in its entirety.

The detected WHF event, or a human-perceptible notification of the detection of the WHF event, may be presented to a user via the user interface 220, such as being displayed on a display screen. Also displayed or otherwise presented to the user via the user interface 220 may include one or more of the sensed physiologic signal, signal metrics, patient heart failure phenotype Px, target phenotype P* recognized from the phenotype bank, and the detection configurations DX*, among other intermediate measurements or computations. The information may be presented in a table, a chart, a diagram, or any other types of textual, tabular, or graphical presentation formats. The presentation of the output information may include audio or other media format. In an example, alerts, alarms, emergency calls, or other forms of warnings may be generated to signal the system user about the detected WHF event.

The optional therapy circuit 250 may be configured to deliver a therapy to the patient in response to the detected WHF event. Examples of the therapy may include electrostimulation therapy delivered to the heart, a nerve tissue, other target tissues, a cardioversion therapy, a defibrillation therapy, or drug therapy including delivering drug to a tissue or organ. In some examples, the therapy circuit 250 may modify an existing therapy, such as adjust a stimulation parameter or drug dosage.

Although the discussion herein focuses on WHF event detection, this is meant only by way of example but not limitation. Systems, devices, and methods discussed in this document may also be suitable for detecting various sorts of diseases or for assessing risk of developing other worsened conditions, such as cardiac arrhythmias, heart failure decompensation, pulmonary edema, pulmonary condition exacerbation, asthma and pneumonia, myocardial infarction, dilated cardiomyopathy, ischemic cardiomyopathy, valvular disease, renal disease, chronic obstructive pulmonary disease, peripheral vascular disease, cerebrovascular disease, hepatic disease, diabetes, anemia, or depression, among others.

FIGS. 3A-3B illustrate generally examples of mapping from various phenotypes to corresponding detection configurations. The phenotype-detection configuration maps 310 and 320 illustrated herein are embodiments of the phenotype-detection configuration map 244 in FIG. 2. As illustrated in FIG. 3A, the phenotype-detection configuration map 310 associates a plurality of pre-determined heart failure phenotypes {P₁, P₂, . . . , P_{N}) into corresponding detection configurations ΔDX₁, DX₂, . . . , DX_{N}}. For example, phenotype Pᵢ may be associated with detection configuration DXᵢ. Each phenotype may include one or more categories of information about patient demographic information, medical history, medication information, or lab test results. Some information categories may further include two or more attributes. A phenotype such as Pᵢ may be defined by multiple patient attributes each having a specified numerical value or a range of values, or a specified categorical value. A detection configuration such as DX₁ may refer to algorithms and parameters used for WHF event detection corresponding to the phenotype Pᵢ, and may include one or more of physiologic signal metrics selection, detection parameter settings, or WHF detection algorithms. Examples of physiologic signal selection may include selecting one or more signal metrics, selectively activating a physiologic sensor for sensing and acquiring respective physiologic signal, selecting particular signal portions when the patient undergoes a particular physical activity level or during a particular time of day, or under other specified conditions. Examples of the detection parameters may include detection threshold values. Examples of the WHF detection algorithms may include different weights assigned to the signal metrics used for establishing a composite index. In an example, if the patient phenotype includes an attribute of significant shortness of breath, the corresponding detection configuration DX may include a larger weight assigned to respiration rate (RR) trend for constructing a composite index for WHF event detection. In another example, if the patient phenotype includes an attribute of significant palpitation, the corresponding detection configuration DX may include a larger weight assigned to heart rate trend for constructing a composite index for WHF event detection. Yet in another example, if the patient phenotype includes an attribute of edema (such as due to long-term standing), the corresponding detection configuration DX may include a larger weight assigned to total thoracic impedance for constructing a composite index for WHF event detection.

FIG. 3B illustrates a phenotype-detection configuration map 320 that associates a plurality of phenotype scores or a score ranges {S₁, S₂, . . . , S_{N}} into corresponding detection configurations {DX₁, DX₂, . . . , DX_{N}}. For example, the phenotype score Sᵢ may be associated with the detection configuration DXᵢ. The phenotype score represents an aggregated risk of WHF. In an example, the phenotype-detection configuration map 320 may be constructed using patient attributes and detection performance data collected from a patient population. The phenotype score Sᵢ may be computed by accumulating individual attribute score for each patient attribute. The attribute score represents a patient attribute satisfying a specific condition (e.g., exceeding a threshold, falling within a value range, or being categorized into a specific category). For example, a patient attribute of “Medication=no beta blocker” is assigned an attribute score of 1, and “Medication=beta blocker” has an attribute score of 0. In another example, a patient attribute of “Sex=male” is assigned an attribute score of 0.2, and “Sex=Female” has an attribute score of 0. The detection configuration that leads to desired detection performance may be associated with the phenotype score or score range. As the phenotype score represents an aggregated risk of WHF based on a multitude of patient attributes, different WHF detection algorithms (or algorithms with different detection threshold values) may be selected based on the phenotype score. For example, between a larger phenotype score Sᵢ and a lower phenotype score Sⱼ (Sᵢ>Sⱼ), the higher phenotype score Si may be mapped to a detection configuration DXᵢ that includes a detection algorithm having a higher sensitivity, such that false negatives or miss of WHF event detection may be reduced. The lower phenotype score Sⱼ may be mapped to a detection configuration DXⱼ that includes a detection algorithm having a higher specificity, such that false positive WHF event detection may be reduced.

In various examples, the phenotype-detection configuration maps 310 or 320 may be updated when additional patient data become available, including information of patient phenotypes and WHF detection performance. The update may be performed periodically, or in response to a user command or a triggering event.

FIG. 4 illustrates a diagram 400 of computing a phenotype score (S_{X}) for the patient heart failure phenotype (P_{X}). The phenotype score may be computed using the classifier circuit 232. By way of example and not limitation, the phenotype as illustrated is characterized by patient attributes including medication information of beta blocker usage, medical history of ischemic heart disease, race as being Caucasian or not, sex as being male or female, and BMI. An attribute score may be assigned to each of the patient attribute based on the categorical value, or numerical value or range. In the example as illustrated, the attribute score may take a value between 0 and 1, where “1” represents a high WHF risk, and “0” represents a low WHF risk. The attribute scores may be accumulated to produce a phenotype score S_{X} corresponding to the patient phenotype Px. The Sx may be mapped to the detection configuration DX*, such as according to the phenotype-detection configuration map 320.

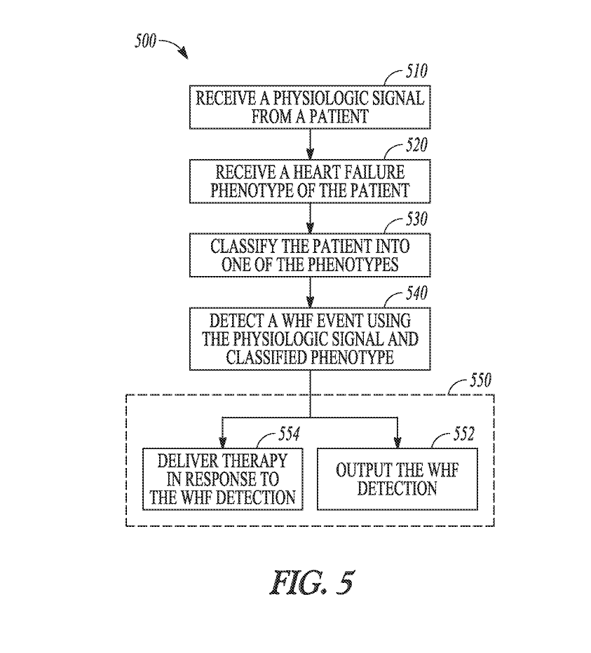

FIG. 5 illustrates generally an example of a method 500 for detecting WHF in a patient based on phenotype classification. The method 500 may be implemented and executed in an ambulatory medical device, such as an implantable or wearable medical device, or in a remote patient management system. In various examples, the method 500 may be implemented in and executed by the AMD 110, one or more devices in the external system 125, or the heart failure monitor system 200 or a modification thereof.

The method 500 commences at step 510, where a physiologic signal may be received from a patient. The physiologic signal may contain information about patient physiologic response to a precipitating event indicative of WEIR Examples of the physiologic signals for detecting WHF may include ECG, EGM, heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. in an example, the physiologic signal may be sensed and acquired using the sensor circuit 210 that is coupled to one or more implantable, wearable, or otherwise ambulatory sensors or electrodes associated with the patient. Alternatively, the sensed physiologic signal may be acquired and stored in a storage device, such as an electronic medical record system, and may be retrieved in response to a user input or triggered by a specific event.

At 520, a heart failure phenotype of the patient may be received. In an example, a user, such as the patient or a clinician, may provide information about the patient heart failure phenotype such as via the user interface 220, or make selections from a plurality of pre-determined heart failure phenotypes. The patient heart failure phenotype may include patient vital signs, patient demographic information, medical history including prior medical, surgical, or treatment, dietary and physical activity patterns, weight, and heart failure comorbid conditions, clinical assessment, lab assessments such as blood urea nitrogen (BUN) level, thiamine pyrophosphate (TPP) level, or other blood chemistry, among other patient attributes.

At 530, the patient may be classified into one of a plurality of heart failure phenotypes based on the received patient heart failure phenotype. In an example, the classification may include a process of searching a phenotype bank for a target heart failure phenotype (P*) that matches the received patient heart failure phenotype (Px) using a pattern recognition method. As previously discussed with reference to FIG. 2, the heart failure phenotype bank may store a plurality of pre-determined heart failure phenotypes {P₁, P₂, . . . , P_{N}}. Each of the heart failure phenotypes may include one or more patient attributes, such as information about patient demographics, medical history, medication intake and dosage, lab tests, among others. To recognize a target phenotype P*, similarity metrics, such as distance measures in the multi-dimensional attribute space between the received patient heart failure phenotype and the pre-determined heart failure phenotypes may be computed. The target phenotype P* may be determined as one with a shortest distance to the patient heart failure phenotype.

Each of the pre-determined heart failure phenotype (Pᵢ) in the phenotype bank may be associated with a detection configuration (DXᵢ). FIG. 3A illustrates an example of such a phenotype-detection configuration map 310. The DXᵢ may include an optimal parameter setting for detecting a WHF event in patients having the same phenotype Pᵢ, such as detection threshold values. The optimal parameter setting for the phenotype Pᵢ may be determined as one that leads to a WHF event detection performance satisfying a specific condition based on data collected patient population data. In an example, the DXᵢ may include a selection of physiologic signal metrics used for WHF event detection, selective activation of a physiologic sensor for sensing and acquiring respective physiologic signal, or selection of particular signal portions such as when the patient undergoes a particular physical activity level or during a particular time of day. In another example, the DXᵢ may include a selection of a WHF detection algorithm for WHF event detection. Examples of the WHF detection algorithms may include different weights assigned to the signal metrics used for establishing a composite index.

By recognizing the target heart failure phenotype (P*) that matches the received patient heart failure phenotype (such as based on the shortest distance to the patient heart failure phenotype in the attribute space), a detection configuration (DX*) corresponding to the target phenotype (P*) may be identified based on the phenotype-detection configuration map. At 540, a WHF event maybe then detected from the received physiologic signal using the detection configuration DX*. Detection of the WHF event may include a comparison of a signal metric to a detection threshold which may be specified in the detection configuration DX*. In some examples, detection of the WHF event may include computing a composite signal index using signal metrics derived from the received one or more physiologic signals, and detecting WHF and generating a WHF alert when the composite signal index exceeds a detection threshold. In some examples, detection of the WHF event may include generating a predictor trend using a difference between short-term values and baseline values. The parameters used for computing the short-term and baseline value may be specified in the detection configuration DX*. The predictor trend indicates temporal changes of the signal metric trend. Alternatively, the predictor trend may be determined using a linear or nonlinear combination of the relative differences between multiple short-term values corresponding to multiple first time windows and multiple baseline values corresponding to multiple second time windows, such as described by Thakur et al., in U.S. Patent Publication 2017/0095160, entitled “PREDICTIONS OF WORSENING HEART FAILURE”, which is herein incorporated by reference in its entirety.

In some examples, the classification of the patient into a particular phenotype at 530 may include a process of computing a patient phenotype score using attributes of the received patient heart failure phenotype. FIG. 3B illustrates an example of the phenotype score-based classification and detection configuration mapping. Each attribute of the patient heart failure phenotype that satisfies a specific condition may be assigned an attribute score. A phenotype score (S_{X}) may be computed for the received patient heart failure phenotype (P_{X}), and the patient may be classified into a phenotype based on the phenotype score S_{X}. The phenotype score or a score range may be mapped to a detection configuration, as illustrated in FIG. 3B. Accordingly, a detection configuration (DX*) corresponding to S_{X} may be identified according to the phenotype-detection configuration map. Detection of WHF event maybe carried out using the detection configuration DX* at 540.

At 550, the detected WHF event, or a human-perceptible notification of the detection of the WHF event, may be presented to a user or a process. At 552, a human-perceptible presentation of the detected WHF ever may be generated, and displayed such as on the user interface 220. Sensed physiologic signal, signal metrics, patient heart failure phenotype P_{X}, target phenotype P* recognized from the phenotype bank, or detection configurations DX*, may also be displayed. The information may be presented in a table, a chart, a diagram, or any other types of textual, tabular, or graphical presentation formats. Hard copies of signals and information related to the WHF event detection may be generated. In an example, alerts, alarms, emergency calls, or other forms of warnings to signal the system user about the WHF event detection may be generated.

Additionally or alternatively, at 554, the detected WHF event may trigger a therapy delivered to the patient, such as using the therapy circuit 250. Examples of the therapy may include electrostimulation therapy delivered to the heart, a nerve tissue, other target tissues, a cardioversion therapy, a defibrillation therapy, or drug therapy. In some examples, an existing therapy may be modified, such as by adjusting a stimulation parameter or drug dosage.

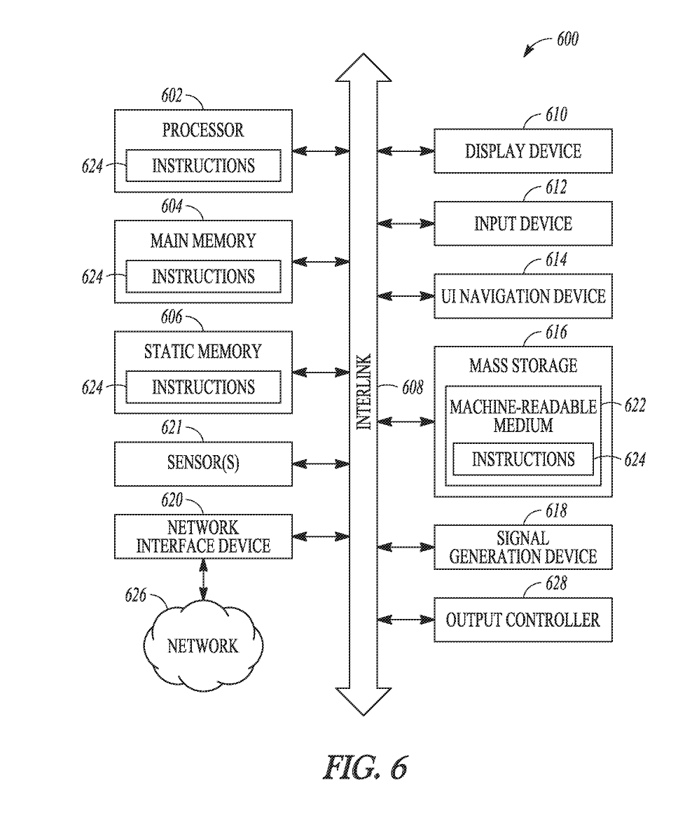

FIG. 6 illustrates generally a block diagram of an example machine 600 upon which any one or more of the techniques (e.g., methodologies) discussed herein may perform. Portions of this description may apply to the computing framework of various portions of the LCP device, the IMD, or the external programmer.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specific operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., universal serial bus (USB), parallel, or other wired or wireless infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine-readable media.

While the machine-readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks,

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for detecting worsening heart failure (WHF) in a patient, comprising:
a signal receiver configured to receive a physiologic signal from the patient;
a phenotype receiver configured to receive a heart failure phenotype of the patient including patient demographic information; and
a processor circuit, including:
a classifier circuit configured to classify the patient into one of a plurality of phenotypes based on the received heart failure phenotype, the plurality of phenotypes each represented by multi-dimensional categorized demographics; and
a detector circuit configured to detect a WHF event using the sensed physiologic signal and the classified phenotype.

2. The system of claim 1, wherein the plurality of phenotypes each further include medical history information.

3. The system of claim 1, wherein the plurality of phenotypes each further include medication information.

4. The system of claim 1, wherein the received heart failure phenotype further includes medical history or medication information of the patient, and the classifier circuit is configured to classify the patient into one of the plurality of phenotypes in response to a change in the medical history or medication of the patient.

5. The system of claim 1, comprising a storage device configured to store a correspondence between the plurality of phenotypes and the corresponding multi-dimensional categorized demographics, wherein the classifier circuit is configured to classify the patient into one of the plurality of phenotypes using the stored correspondence.

6. The system of claim 1, wherein the classifier circuit is configured to determine similarity metrics between the received heart failure phenotype and each of the plurality of phenotypes, and to classify the patient into one of the plurality of phenotypes based on the similarity metrics.

7. The system of claim 1, wherein the classifier circuit is configured to compute a patient phenotype score using a combination of numerical values respectively assigned to the received patient demographic information, and to classify the patient into one of the plurality of phenotypes based on the computed patient phenotype score.

8. The system of claim 1, wherein the detector circuit is configured to identify a detection algorithm based on the classified phenotype, and to detect the WHF event using the identified detection algorithm and the sensed physiologic signal.

9. The system of claim 1, wherein the detector circuit is configured to compute a composite signal metric using the sensed physiologic signal, and to detect the WHF event using the composite signal metric.

10. The system of claim 9, wherein the detector circuit is configured to adjust a threshold value based on the classified phenotype threshold value, and to detect the WHF event using a comparison of the composite signal metric to the adjusted threshold value.

11. The system of claim 9, wherein the detector circuit is configured to:
generate a plurality of signal metrics from the sensed physiologic signal;
assign weight factors to the plurality of signal metrics based on the classified phenotype; and
compute the composite signal metric using a weighted combination of the plurality of the signal metrics respectively scaled by the assigned weight factors.

12. The system of claim 11, wherein the detector circuit is configured to assign weight factors including to:
increase a weight factor to a respiration rate metric if the classified phenotype includes an attribute of significant shortness of breath;
increase a weight factor to a heart rate metric if the classified phenotype includes an attribute of palpitation; or
increase a weight factor to a total thoracic impedance metric if the classified phenotype includes an attribute of edema.

13. The system of claim 1, comprising a therapy circuit configured to generate and deliver a heart failure therapy in response to the detection of the WHF event.

14. A method for detecting worsening heart failure (WHF) in a patient using a medical system, comprising:
receiving a physiologic signal from the patient;
receiving a heart failure phenotype of the patient including patient demographic information; and
classifying the patient into one of a plurality of phenotypes based on the received heart failure phenotype, the plurality of phenotypes each represented by multi-dimensional categorized demographics; and
detecting a WHF event using the sensed physiologic signal and the classified phenotype.

15. The method of claim 14, wherein the received heart failure phenotype further includes medical history or medication information of the patient, and the classifier circuit is configured to classify the patient into one of the plurality of phenotypes in response to a change in the medical history or medication of the patient.

16. The method of claim 14, comprising determining similarity metrics between the received heart failure phenotype and each of the plurality of phenotypes, wherein classifying the patient into one of the plurality of phenotypes is based on the similarity metrics.

17. The method of claim 14, comprising computing a patient phenotype score using the received heart failure phenotype, wherein classifying the patient into one of the plurality of phenotypes is based on the computed patient phenotype score.

18. The method of claim 14, comprising computing a composite signal metric using the sensed physiologic signal, and wherein detecting the WHF event is based on the composite signal metric.

19. The method of claim 18, comprising adjusting a threshold value based on the classified phenotype threshold value, wherein detecting the WHF event includes using a comparison of the composite signal metric to the adjusted threshold value.

20. The method of claim 18, comprising:
generating a plurality of signal metrics from the sensed physiologic signal; and
assigning weight factors to the plurality of signal metrics based on the classified phenotype;
wherein computing the composite signal metric includes a weighted combination of the plurality of the signal metrics respectively scaled by the assigned weight factors.
